(19) United States
(12) Patent Application Publication
(10) Pub. No.: US 2018/0305313 A1
(43) Pub. Date: Oct. 25, 2018

(54) COLORED PHOTOSENSITIVE RESIN COMPOSITION AND LIGHT SHIELDING SPACER PREPARED THEREFROM

(71) Applicant: Rohm and Haas Electronic Materials Korea Ltd., Cheonan (KR)

(72) Inventors: Seok-Bong Park, Yongin (KR); Gyung-Sik Choi, Hwaseong (KR); Yeonok Kim, Hwaseong (KR)

(21) Appl. No.: 15/769,875

(22) PCT Filed: Sep. 23, 2016
(86) PCT No.: PCT/KR2016/010629

§ 371 Date: Apr. 20, 2018

(30) Foreign Application Priority Data
Nov. 4, 2015 (KR) 10-2015-0154797
Sep. 22, 2016 (KR) 10-2016-0121543

(51) Int. Cl. C07D 209/82; C07D 251/12; C07D 317/10; C07D 405/12; G03F 7/00; G03F 7/029; G03F 7/031; G03F 7/038; G03F 7/105

(52) U.S. Cl. CPC C07D 209/82; C07D 251/12; C07D 317/10; C07D 405/12; G03F 7/105; G03F 7/029; G03F 7/031; G03F 7/0388; G03F 7/0007

(57) ABSTRACT

Disclosed herein is a colored photosensitive resin composition including a copolymer, an epoxy resin compound or a compound derived therefrom, a polymerizable compound, a photoinitiator, and a colorant, wherein the photoinitiator includes an oxime compound and a triazine compound. The composition, when formed into a cured film, may facilitate the fabrication of necessary height difference and satisfy the requirements of sensitivity and an exposure margin for light shielding spacers, and, thus, is useful as a material for manufacturing a light shielding spacer such as a black column spacer used in various electronic parts including the panels of an LCD and an OLED display.

### TECHNICAL FIELD

The present invention relates to a colored photosensitive resin composition suitable as a material for forming a passivation layer, an interlayer dielectric, a spacer, a light shielding part, etc. employed in a panel of a liquid crystal display (LCD) or an organic light-emitting diode (OLED) display, and a light shielding spacer prepared from the composition.

### BACKGROUND ART

Recently, a spacer formed from a photosensitive resin composition is employed in order to maintain the distance between the upper and lower transparent substrates in a liquid crystal cell of an LCD. In an LCD, which is an electro-optical device driven by a voltage applied to a liquid crystal material injected into a constant gap between two transparent substrates, it is very crucial to maintain the gap between the two substrates constant. If the gap between the transparent substrates is not constant, the voltage applied thereto as well as the transmittance of light penetrating this area may vary, resulting in defects of spatially non-uniform luminance. According to a recent demand for large LCD panels, it is even more crucial to maintain a constant gap between two transparent substrates.

A spacer may be formed by coating a photosensitive resin composition onto a substrate and exposing the coated substrate to ultraviolet light, etc., by using a mask, followed by development thereof. Recently, efforts of using a light shielding material for a spacer have been made; accordingly, various colored photosensitive resin compositions have been actively developed.

In this regard, attempts have been made to improve the chemical resistance, developability, exposure margin, etc. of a spacer by using colored photosensitive resin compositions in which various photoinitiators are employed.

For example, Korean Registration Patent No. 10-0842168 discloses a photosensitive resin composition for accomplishing minute patterns by including at least two kinds of acetophenone photoinitiators and non-imidazole photoinitiators. However, the photosensitive resin composition is still insufficient in terms of chemical resistance and exposure margin.

Meanwhile, recently, attempts have been made to simplify manufacturing processes by developing a black column spacer in which a column spacer and a black matrix are integrated into one module. A colored photosensitive resin composition used for the manufacture of such black column spacer is required to facilitate the fabrication of necessary height difference as well as have satisfactory sensitivity and an exposure margin at the same time.

### DISCLOSURE OF INVENTION

### Technical Problem

Accordingly, it is an object of the present invention to provide a colored photosensitive resin composition that may facilitate the fabrication of necessary height difference and satisfy the requirements of sensitivity and an exposure margin for the manufacture of a light shielding spacer such as a black column spacer.

### Solution to Problem

In accordance with one aspect of the present invention, there is provided a colored photosensitive resin composition including (a) a copolymer; (b) an epoxy resin compound or a compound derived therefrom; (c) a polymerizable compound; (d) a photoinitiator; and (e) a colorant, wherein the photoinitiator includes a compound of the following Formula 1 and a compound of the following Formula 2.

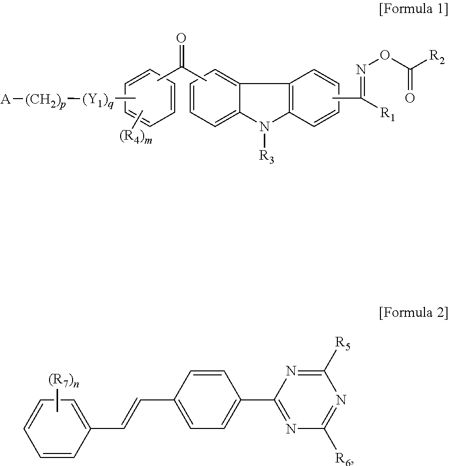

wherein, in Formula 1, R₁ to R₄ are each independently hydrogen, deuterium, halogen, substituted or unsubstituted C₁₋₁₂ alkyl, substituted or unsubstituted C₂₋₁₂ alkenyl, substituted or unsubstituted halo-C₁₋₁₂ alkyl, substituted or unsubstituted C₆₋₁₂ aryl, substituted or unsubstituted C₃₋₁₂ cycloalkyl, substituted or unsubstituted C₁₋₁₂ alkoxy, or C₁₋₁₂ ester; A is substituted or unsubstituted 5- to 12-membered heteroaryl, or substituted or unsubstituted 5- to 7-membered heterocycloalkyl; R₁ to R₄ and substituents of A are each independently at least one selected from the group consisting of halogen, halo-C₁₋₁₂ alkyl, C₁₋₁₂ alkyl, C₂₋₁₂ alkenyl, C₆₋₁₂ aryl, C₃₋₁₂ cycloalkyl, C₁₋₁₂ alkoxy, carboxy, nitro and hydroxy; Y₁ is —O—, —S—, or —Se—; m is an integer of 0 to 4; in the case where m is an integer of 2 or more, R₄s are the same or different from each other; p is an integer of 0 to 5; and q is 0 or 1, and in Formula 2, R₅ and R₆ are halomethyl; R₇ is each independently C₁₋₄ alkyl or C₁₋₄ alkoxy; and n is an integer of 0 to 3.

In addition, there is provided a light shielding spacer formed by curing the above colored photosensitive resin composition.

### Advantageous Effects of Invention

The colored photosensitive resin composition of the present invention, when formed into a cured film, may facilitate the fabrication of necessary height difference and satisfy the requirements of sensitivity and an exposure margin for light shielding spacers, and, thus, is useful as a material for manufacturing a light shielding spacer such as a black column spacer used in various electronic parts including the panels of an LCD and an OLED display.

### BRIEF DESCRIPTION OF DRAWINGS

FIG. 1 is a schematic diagram of an embodiment of the cross-section of a light shielding spacer (black column spacer).

### EXPLANATION ON SYMBOLS

A: Thickness of column spacer part
B: Thickness of black matrix part
C: Critical dimension (CD) of column spacer part

### BEST MODE FOR CARRYING OUT THE INVENTION

The photosensitive resin composition according to the present invention may include (a) a copolymer, (b) an epoxy resin compound or a compound derived therefrom, (c) a polymerizable compound, (d) a photoinitiator, and (e) a colorant and may further include (f) a surfactant, (g) a silane coupling agent, and/or (h) a solvent, if desired.

In the present specification, “(meth)acryl” means “acryl” and/or “methacryl,” and “(meth)acrylate” means “acrylate” and/or “methacrylate.”

Hereinafter, each component of the colored photosensitive resin composition of the present invention will be explained in detail.

(a) Copolymer

The copolymer used in the present invention may include (a-1) a structural unit derived from an ethylenically unsaturated carboxylic acid, an ethylenically unsaturated carboxylic anhydride, or a combination thereof, and (a-2) a structural unit derived from an ethylenically unsaturated compound containing an aromatic ring, and may additionally include (a-3) a structural unit derived from an ethylenically unsaturated compound different from (a-1) and (a-2).

The copolymer is an alkali-soluble resin for having developability in a development step and may also play the role of a base for forming a coated film thereon and a structure for attaining a final pattern.

(a-1) Structural Unit Derived from an Ethylenically Unsaturated Carboxylic Acid, an Ethylenically Unsaturated Carboxylic Anhydride, or a Combination Thereof

In the present invention, the structural unit (a-1) is derived from an ethylenically unsaturated carboxylic acid, an ethylenically unsaturated carboxylic anhydride, or a combination thereof. The ethylenically unsaturated carboxylic acid or the ethylenically unsaturated carboxylic anhydride is a polymerizable unsaturated monomer containing at least one carboxyl group in a molecule. Preferable examples thereof may include an unsaturated monocarboxylic acid such as (meth)acrylic acid, crotonic acid, alpha-chloroacrylic acid, and cinnamic acid; an unsaturated dicarboxylic acid and an anhydride thereof such as maleic acid, maleic anhydride, fumaric acid, itaconic acid, itaconic anhydride, citraconic acid, citraconic anhydride, and mesaconic acid; an unsaturated polycarboxylic acid of trivalence or more and an anhydride thereof; and a mono[(meth)acryloyloxyalkyl] ester of a polycarboxylic acid of divalence or more such as mono[2-(meth)acryloyloxyethyl] succinate, mono[2-(meth)acryloyloxyethyl] phthalate, and the like. The structural unit derived from the above compounds may be included in the copolymer alone or as a combination of two or more.

The amount of the structural unit (a-1) may be 5 to 65 mole %, and preferably 10 to 50 mole %, based on the total moles of the structural units constituting the copolymer. Within this amount range, the developability may be easily maintained.

(a-2) Structural Unit Derived from an Ethylenically Unsaturated Compound Containing an Aromatic Ring

The structural unit (a-2) is derived from an ethylenically unsaturated compound containing an aromatic ring, and preferable examples of the ethylenically unsaturated compound containing an aromatic ring may include phenyl (meth)acrylate, benzyl (meth)acrylate, 2-phenoxyethyl (meth)acrylate, phenoxy diethylene glycol (meth)acrylate, p-nonylphenoxy polyethylene glycol (meth)acrylate, p-nonylphenoxy polypropylene glycol (meth)acrylate, tribromophenyl (meth)acrylate; styrene; styrene containing an alkyl substituent such as methylstyrene, dimethylstyrene, trimethylstyrene, ethylstyrene, diethylstyrene, triethylstyrene, propylstyrene, butylstyrene, hexylstyrene, heptylstyrene, and octylstyrene; styrene containing halogen such as fluorostyrene, chlorostyrene, bromostyrene, and iodostyrene; styrene containing an alkoxy substituent such as methoxystyrene, ethoxystyrene, and propoxystyrene; 4-hydroxystyrene, p-hydroxy-α-methylstyrene, acetylstyrene; vinyltoluene, divinylbenzene, vinylphenol, o-vinylbenzyl methyl ether, m-vinylbenzyl methyl ether, p-vinylbenzyl methyl ether, o-vinylbenzyl glycidyl ether, m-vinylbenzyl glycidyl ether, p-vinylbenzyl glycidyl ether, and the like.

The structural unit derived from the above exemplified compounds may be included in the copolymer alone or as a combination of two or more.

Among the above compounds, the styrene compounds may be preferably used in consideration of polymerization property.

The amount of the structural unit (a-2) may be 2 to 70 mole %, and preferably 5 to 60 mole %, based on the total moles of the structural units constituting the copolymer. Within this amount range, favorable chemical resistance may be attained.

(a-3) Structural Unit Derived from an Ethylenically Unsaturated Compound Different from (a-1) and (a-2)

In addition to (a-1) and (a-2), the copolymer used in the present invention may additionally include a structural unit derived from an ethylenically unsaturated compound different from (a-1) and (a-2).

The ethylenically unsaturated compound different from (a-1) and (a-2) may include an unsaturated carboxylic acid ester such as methyl (meth)acrylate, ethyl (meth)acrylate, butyl (meth)acrylate, dimethylaminoethyl (meth)acrylate, isobutyl (meth)acrylate, t-butyl (meth)acrylate, cyclohexyl (meth)acrylate, ethylhexyl (meth)acrylate, tetrahydrofurfuryl (meth)acrylate, hydroxyethyl (meth)acrylate, 2-hydroxypropyl (meth)acrylate, 2-hydroxy-3-chloropropyl (meth)acrylate, 4-hydroxybutyl (meth)acrylate, glycerol (meth)acrylate, methyl α-hydroxymethylacrylate, ethyl α-hydroxymethylacrylate, propyl α-hydroxymethylacrylate, butyl α-hydroxymethylacrylate, 2-methoxyethyl (meth)acrylate, 3-methoxybutyl (meth)acrylate, ethoxy diethylene glycol (meth)acrylate, methoxy triethylene glycol (meth)acrylate, methoxy tripropylene glycol (meth)acrylate, poly(ethylene glycol) methyl ether (meth)acrylate, tetrafluoropropyl (meth)acrylate, 1,1,1,3,3,3-hexafluoroisopropyl (meth)acrylate, octafluoropentyl (meth)acrylate, heptadecafluorodecyl (meth)acrylate, isobornyl (meth)acrylate, dicyclopentanyl (meth)acrylate, dicyclopentenyl (meth)acrylate, dicyclopentanyloxyethyl (meth)acrylate, and dicyclopentenyloxyethyl (meth)acrylate; a tertiary amine containing an N-vinyl group such as N-vinyl pyrrolidone, N-vinyl carbazole and N-vinyl morpholine; an unsaturated ether such as vinyl methyl ether and vinyl ethyl ether; an ethylenically unsaturated compound containing an epoxy group such as glycidyl (meth)acrylate, 3,4-epoxybutyl (meth)acrylate, 4,5-epoxypentyl (meth)acrylate, 5,6-epoxyhexyl (meth)acrylate, 6,7-epoxyheptyl (meth)acrylate, 2,3-epoxycyclopentyl (meth)acrylate, 3,4-epoxycyclohexyl (meth)acrylate, α-ethyl glycidyl acrylate, α-n-propyl glycidyl acrylate, α-n-butyl glycidyl acrylate, N-(4-(2,3-epoxypropoxy)-3,5-dimethylbenzyl)acrylamide, N-(4-(2,3-epoxypropoxy)-3,5-dimethylphenylpropyl)acrylamide, 4-hydroxybutyl (meth)acrylate glycidyl ether, allyl glycidyl ether, and 2-methylallyl glycidyl ether; an unsaturated imide such as N-phenylmaleimide, N-(4-chlorophenyl)maleimide, N-(4-hydroxyphenyl)maleimide and N-cyclohexylmaleimide, and the like.

The structural unit derived from the above exemplified compounds may be included in the copolymer alone or as a combination of two or more.

Preferably, the structural unit derived from the ethylenically unsaturated compound containing an epoxy group and/or the unsaturated imide may be used, and glycidyl (meth)acrylate, 4-hydroxybutyl (meth)acrylate glycidyl ether, and/or a structural unit derived from N-substituted maleimide may be more preferable in consideration of the improvement in copolymerization properties and the intensity of an insulating film.

The amount of the structural unit (a-3) may be 10 to 80 mole %, and preferably 20 to 75 mole %, based on the total moles of the structural units constituting the copolymer. Within this amount range, the storage stability of a colored photosensitive resin composition may be maintained and a residual film thickness may be improved.

The copolymer having the structural units (a-1) to (a-3) may include a copolymer of (meth)acrylic acid/styrene, a copolymer of (meth)acrylic acid/benzyl (meth)acrylate, a copolymer of (meth)acrylic acid/styrene/methyl (meth)acrylate, a copolymer of (meth)acrylic acid/styrene/methyl (meth)acrylate/glycidyl (meth)acrylate, a copolymer of (meth)acrylic acid/styrene/methyl (meth)acrylate/glycidyl (meth)acrylate/N-phenylmaleimide, a copolymer of (meth)acrylic acid/styrene/methyl (meth)acrylate/glycidyl (meth)acrylate/N-cyclohexylmaleimide, a copolymer of (meth)acrylic acid/styrene/n-butyl (meth)acrylate/glycidyl (meth)acrylate/N-phenylmaleimide, a copolymer of (meth)acrylic acid/styrene/glycidyl (meth)acrylate/N-phenylmaleimide, a copolymer of (meth)acrylic acid/styrene/4-hydroxybutyl (meth)acrylate glycidyl ether/N-phenylmaleimide, and the like.

At least one or at least two of the copolymers may be included in the colored photosensitive resin composition.

The weight average molecular weight (Mw) of the copolymer may be in the range of 3,000 to 50,000, and preferably 5,000 to 40,000, when determined by gel permeation chromatography (eluent: tetrahydrofuran) referenced to polystyrene. Within this range, the improved adhesiveness to a substrate, physical/chemical properties and viscosity may be advantageously obtained.

The copolymer may be used in an amount ratio of 0.5 to 60 wt %, and preferably 5 to 50 wt %, based on the total weight of the solid content of the colored photosensitive resin composition (i.e., weight excluding solvents). Within this range, the composition may produce a film having a good pattern profile after development and improved properties such as chemical resistance.

The copolymer may be prepared by injecting to a reactor a molecular weight regulator, a radical polymerization initiator, a solvent, and the structural units (a-1) to (a-3), further injecting nitrogen thereto, and then stirring slowly for polymerizing.

The molecular weight regulator may be a mercaptan compound such as butyl mercaptan, and octyl mercaptan, or an α-methylstyrene dimer, but is not limited thereto.

The radical polymerization initiator may be an azo compound such as 2,2′-azobisisobutyronitrile, 2,2′-azobis(2,4-dimethylvaleronitrile), and 2,2′-azobis(4-methoxy-2,4-dimethylvaleronitrile); or a peroxide such as benzoyl peroxide, lauryl peroxide, t-butyl peroxypivalate and 1,1-bis(t-butylperoxy)cyclohexane, but is not limited thereto. The radical polymerization initiator may be used alone or as a mixture of two or more.

Also, the solvent may be any conventional solvents commonly used in the preparation of a copolymer and may include, e.g., propylene glycol monomethyl ether acetate (PGMEA).

(b) Epoxy Resin Compound or a Compound Derived Therefrom

The colored photosensitive resin composition of the present invention includes an epoxy resin compound or a compound derived therefrom.

Preferably, the epoxy resin compound or the compound derived therefrom may have a cardo backbone structure.

The weight average molecular weight (Mw) of the epoxy resin compound or the compound derived therefrom may be in the range of 400 to 10,000 when determined by gel permeation chromatography referenced to polystyrene.

Preferably, the epoxy resin compound or the compound derived therefrom may be an epoxy resin compound having a cardo backbone structure, which is represented by the following Formula 3.

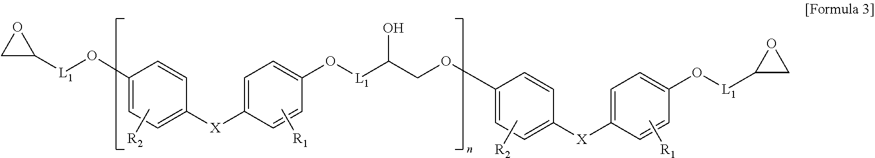

In Formula 3, X is each independently

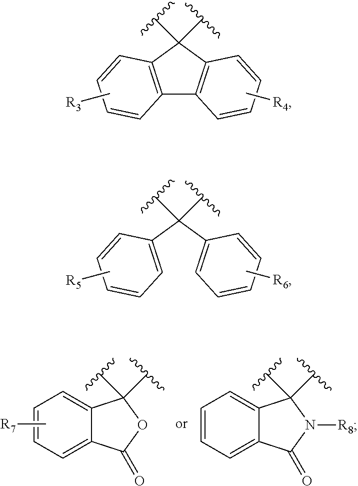

L¹ is each independently a C₁₋₁₀ alkylene group, a C₃₋₂₀ cycloalkylene group, or a C₁₋₁₀ alkyleneoxy group; R₁ to R₇ are each independently H, a C₁₋₁₀ alkyl group, a C₁₋₁₀ alkoxy group, a C₂₋₁₀ alkenyl group, or a C₆₋₁₄ aryl group; R₈ is H, methyl, ethyl, CH₃CHCl—, CH₃CHOH—, CH₂═CHCH₂—, or phenyl; and n is an integer from 0 to 10.

Preferable examples of the C₁₋₁₀ alkylene group may include methylene, ethylene, propylene, isopropylene, butylene, isobutylene, sec-butylene, t-butylene, pentylene, isopentylene, t-pentylene, hexylene, heptylene, octylene, isooctylene, t-octylene, 2-ethylhexylene, nonylene, isononylene, decylene, isodecylene, and the like. Preferable examples of the C₃₋₂₀ cycloalkylene group may include cyclopropylene, cyclobutylene, cyclopentylene, cyclohexylene, cycloheptylene, decalinylene, adamantylene, and the like. Preferable examples of the C₁₋₁₀ alkyleneoxy group may include methyleneoxy, ethyleneoxy, propyleneoxy, butyleneoxy, sec-butyleneoxy, t-butyleneoxy, pentyleneoxy, hexyleneoxy, heptyleneoxy, octyleneoxy, 2-ethyl-hexyleneoxy, and the like. Preferable examples of the C₁₋₁₀ alkyl group may include methyl, ethyl, propyl, isopropyl, butyl, isobutyl, sec-butyl, t-butyl, pentyl, isopentyl, t-pentyl, hexyl, heptyl, octyl, isooctyl, t-octyl, 2-ethylhexyl, nonyl, isononyl, decyl, isodecyl, and the like. Preferable examples of the C₁₋₁₀ alkoxy group may include methoxy, ethoxy, propoxy, butyloxy, sec-butoxy, t-butoxy, pentoxy, hexyloxy, heptoxy, octyloxy, 2-ethyl-hexyloxy, and the like. Preferable examples of the C₂₋₁₀ alkenyl group may include vinyl, allyl, butenyl, propenyl, and the like. Preferable examples of the C₆₋₁₄ aryl group may include phenyl, tolyl, xylyl, naphthyl, and the like.

In a preferred example, the epoxy resin compound having the cardo backbone structure may be prepared through the synthesis route of below:

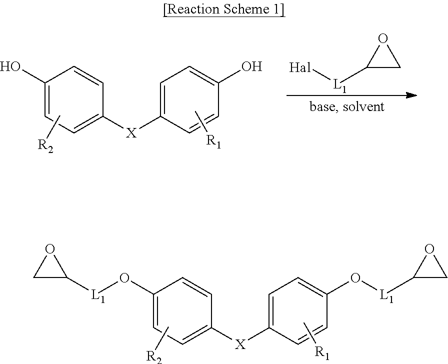

In Reaction Scheme 1, Hal is halogen; and X, R₁, R₂ and L₁ are the same as defined in Formula 3.

The compound derived from the epoxy resin having the cardo backbone structure may be obtained by reacting the epoxy resin having the cardo backbone structure with an unsaturated basic acid to produce an epoxy adduct and then reacting the epoxy adduct thus obtained with a polybasic acid anhydride, or by further reacting the product thus obtained with a monofunctional or polyfunctional epoxy compound. Any unsaturated basic acid known in the art, e.g., acrylic acid, methacrylic acid, crotonic acid, cinnamic acid, sorbic acid, and the like, may be used. Any polybasic acid anhydride known in the art, e.g., succinic anhydride, maleic anhydride, trimellitic anhydride, pyromellitic anhydride, 1,2,4,5-cyclohexane tetracarboxylic dianhydride, hexahydrophthalic anhydride, and the like, may be used. Any monofunctional or polyfunctional epoxy compound known in the art, e.g., glycidyl methacrylate, methyl glycidyl ether, ethyl glycidyl ether, propyl glycidyl ether, isopropyl glycidyl ether, butyl glycidyl ether, isobutyl glycidyl ether, bisphenol Z glycidyl ether, and the like, may be used.

In a preferred example, the compound derived from the epoxy resin having the cardo backbone structure may be prepared through the synthesis route of below:

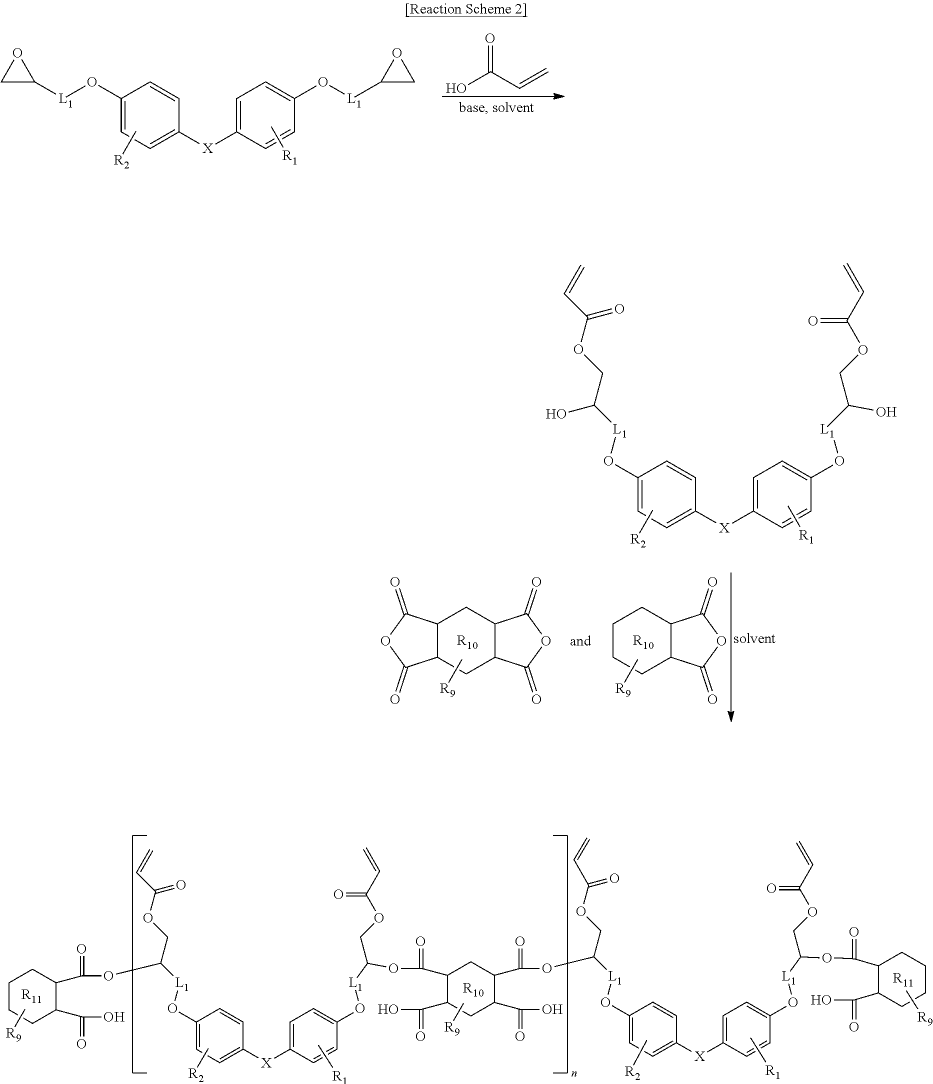

In Reaction Scheme 2, R₉ is each independently H, a C₁₋₁₀ alkyl group, a C₁₋₁₀ alkoxy group, a C₂₋₁₀ alkenyl group, or a C₆₋₁₄ aryl group; R₁₀ and R₁₁ are each independently a saturated or unsaturated C₆ aliphatic ring, or a benzene ring; n is an integer from 1 to 10; and X, R₁, R₂ and L₁ are the same as defined in Formula 3.

When the epoxy resin compound having the cardo backbone structure or the compound derived therefrom is used, the cardo backbone structure may improve the adhesiveness of a cured material to a substrate, alkaline resistance, processability, strength, and the like. Further, an image having a fine resolution may be formed in a pattern once an uncured part is removed upon development.

The amount of the epoxy resin compound or the compound derived therefrom may be 1 to 70 wt %, and preferably 5 to 50 wt %, based on the total amount of the solid content of the colored photosensitive resin composition (i.e., the weight excluding solvents). Within the range, the resolution and chemical resistance may be improved. Further, the pattern profile may be maintained well, and a constant height difference between patterns may be favorably obtained within a desired margin width (i.e., allowable width).

(c) Polymerizable Compound

The polymerizable compound used in the present invention may be any compound that may be polymerized by the action of a polymerization initiator, and may be a polyfunctional monomer, oligomer or polymer commonly used in the preparation of a colored photosensitive resin composition.

More preferably, the polymerizable compound may include a monofunctional or polyfunctional ester compound of acrylic acid or methacrylic acid having at least one ethylenically unsaturated double bond, and may preferably include a polyfunctional compound having at least two functional groups in consideration of chemical resistance.

The polymerizable compound may be selected from the group consisting of ethylene glycol di(meth)acrylate, propylene glycol di(meth)acrylate, diethylene glycol di(meth)acrylate, triethylene glycol di(meth)acrylate, 1,6-hexanediol di(meth)acrylate, polyethylene glycol di(meth)acrylate, polypropylene glycol di(meth)acrylate, glycerin tri(meth)acrylate, trimethylolpropane tri(meth)acrylate, pentaerythritol tri(meth)acrylate, a monoester of pentaerythritol tri(meth)acrylate and succinic acid, pentaerythritol tetra(meth)acrylate, dipentaerythritol penta(meth)acrylate, dipentaerythritol hexa(meth)acrylate, a monoester of dipentaerythritol penta(meth)acrylate and succinic acid, caprolactone modified dipentaerythritol hexa(meth)acrylate, pentaerythritol triacrylate-hexamethylene diisocyanate (a reaction product of pentaerythritol triacrylate and hexamethylene diisocyanate), tripentaerythritol hepta(meth)acrylate, tripentaerythritol octa(meth)acrylate, bisphenol A epoxyacrylate, and ethylene glycol monomethyl ether acrylate, and a mixture thereof, but is not limited thereto.

Examples of a commercially available polymerizable compound may include (i) monofunctional (meth)acrylate such as Aronix M-101, M-111, and M-114 manufactured by Toagosei Co., Ltd., KAYARAD T4-110S, and T4-120S manufactured by Nippon Kayaku Co., Ltd., and V-158, and V-2311 manufactured by Osaka Yuki Kayaku Kogyo Co., Ltd.; (ii) bifunctional (meth)acrylate such as Aronix M-210, M-240, and M-6200 manufactured by Toagosei Co., Ltd., KAYARAD HDDA, HX-220, and R-604 manufactured by Nippon Kayaku Co., Ltd., and V-260, V-312, and V-335 HP manufactured by Osaka Yuki Kayaku Kogyo Co., Ltd.; and (iii) tri and more functional (meth)acrylate such as Aronix M-309, M-400, M-403, M-405, M-450, M-7100, M-8030, M-8060, and TO-1382 manufactured by Toagosei Co., Ltd., KAYARAD TMPTA, DPHA, DPHA-40H, DPCA-20, DPCA-30, DPCA-60, and DPCA-120 manufactured by Nippon Kayaku Co., Ltd., and V-295, V-300, V-360, V-OPT, V-3PA, V-400, and V-802 manufactured by Osaka Yuki Kayaku Kogyo Co., Ltd.

The amount of the polymerizable compound may be 1 to 60 wt %, and preferably 5 to 45 wt % based on the total solid content of the colored photosensitive resin composition (i.e., the weight excluding solvents). Within this range, a pattern may be readily formed, and defects of a pattern profile such as scum may not be generated at a terminal part during development.

(d) Photoinitiator

The photoinitiator used in the present invention includes an oxime photoinitiator (oxime ester photoinitiator) and a triazine photoinitiator.

The oxime photoinitiator is a compound represented by the following Formula 1.

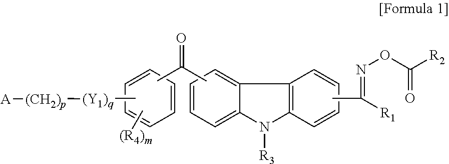

In Formula 1, R₁ to R₄ are each independently hydrogen, deuterium, halogen, substituted or unsubstituted C₁₋₁₂ alkyl, substituted or unsubstituted C₂₋₁₂ alkenyl, substituted or unsubstituted halo-C₁₋₁₂ alkyl, substituted or unsubstituted C₆₋₁₂ aryl, substituted or unsubstituted C₃₋₁₂ cycloalkyl, substituted or unsubstituted C₁₋₁₂ alkoxy, or C₁₋₁₂ ester; A is substituted or unsubstituted 5- to 12-membered heteroaryl, or substituted or unsubstituted 5- to 7-membered heterocycloalkyl; R₁ to R₄ and substituents of A are each independently at least one selected from the group consisting of halogen, halo-C₁₋₁₂ alkyl, C₁₋₁₂ alkyl, C₂₋₁₂ alkenyl, C₆₋₁₂ aryl, C₃₋₁₂ cycloalkyl, C₁₋₁₂ alkoxy, carboxy, nitro and hydroxy; Y₁ is —O—, —S—, or —Se—; m is an integer of 0 to 4; in the case where m is an integer of 2 or more, R₄s are the same or different from each other; p is an integer of 0 to 5; and q is 0 or 1.

Here, the 5- to 12-membered heteroaryl, or the 5- to 7-membered heterocycloalkyl each independently includes at least one heteroatom selected from N, S, and O.

In addition, the C₁₋₁₂ ester means a hydrocarbon group having 1 to 12 carbon atoms and containing an ester group (—C(═O)—O—).

Particularly, the compound of Formula 1 may be represented by the following Formula 1a.

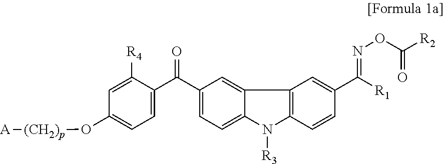

In Formula 1a, R₁ to R₄, p and A are the same as defined in Formula 1.

Particularly, the compound of Formula 1 may be represented by the following Formula 1b.

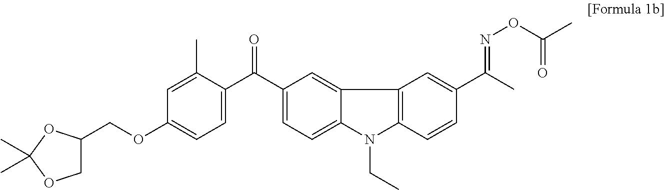 [SMILES: CCn1c2ccc(C(=O)c3ccc(OCC4COC(C)(C)O4)cc3C)cc2c2cc(/C(C)=N/OC(C)=O)ccc21]

The triazine photoinitiator is a compound represented by the following Formula 2.

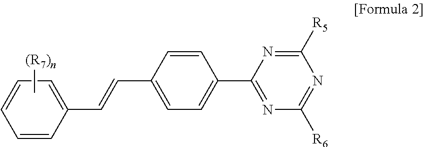

In Formula 2, R₅ and R₆ are halomethyl; R₇ is each independently C₁₋₄ alkyl or C₁₋₄ alkoxy; and n is an integer of 0 to 3.

Particularly, the compound of Formula 2 may be represented by the following Formula 2a.

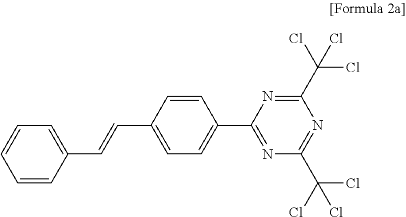 [SMILES: ClC(Cl)(Cl)c1nc(-c2ccc(/C=C/c3ccccc3)cc2)nc(C(Cl)(Cl)Cl)n1]

The oxime photoinitiator of Formula 1 is a highly sensitive initiator reacting at a short wavelength. In the case where the oxime photoinitiator is used alone, although the sensitivity of a colored photosensitive resin composition may be improved, an exposure margin may be deteriorated, and it may be difficult to form a height difference required for a black column spacer, etc. On the other hand, the triazine photoinitiator of Formula 2 is an initiator reacting at a long wavelength and in the case where the triazine photoinitiator is used alone, although the exposure margin of a colored photosensitive resin composition may be favorable, the sensitivity may be deteriorated, thereby decreasing the productivity of a cured film. In the present invention, the combined use of the oxime photoinitiator and the triazine photoinitiator as photoinitiators facilitates the fabrication of a height difference required for a black column spacer, etc. and improves both the sensitivity and the exposure margin of a colored photosensitive resin composition.

Respective amounts of the oxime photoinitiator of Formula 1 and the triazine photoinitiator of Formula 2 may be 0.01 to 10 wt %, and preferably 0.2 to 5 wt %, based on the total solid content of the colored photosensitive resin composition (i.e., the weight excluding solvents).

In this case, the compound of Formula 1 and the compound of Formula 2 may have the weight ratio of 2:8 to 8:2, preferably 2.5:7.5 to 7.5:2.5, and more preferably 3:7 to 7:3. Within these ranges, the composition may be sufficiently cured by light exposure, thereby advantageously achieving excellent sensitivity and exposure margin.

The colored photosensitive resin composition of the present invention may further include another photoinitiator, which may be any known photoinitiator.

The additional photoinitiator may be selected from the group consisting of an acetophenone compound, a non-imidazole compound, an onium salt compound, a benzoin compound, a benzophenone compound, a diketone compound, an α-diketone compound, a polynuclear quinone compound, a thioxanthone compound, a diazo compound, an imidesulfonate compound, a carbazole compound, a sulfonium borate compound, and a mixture thereof.

The photoinitiator may be included in an amount of 0.02 to 20 wt %, and preferably 0.2 to 10 wt %, based on the total solid content of the colored photosensitive resin composition (i.e., the weight excluding solvents). Within this range, the resin composition may be sufficiently cured by light exposure to easily obtain a spacer pattern, and a spacer thus formed may have sufficient adhesiveness to a substrate during development.

(e) Colorant

A colorant is added in the colored photosensitive resin composition of the present invention to impart light shielding properties.

The colorant used in the present invention may be a mixture of two or more inorganic or organic colorants, and preferably has high chromogenic properties and heat resistance. Particularly, a mixture of two or more organic colorants may be favorably used for preventing light leakage through a black matrix and for securing transmittance for mask alignment.

In addition, the colorant may include a black colorant and a blue colorant. The black colorant may be a black inorganic colorant and/or a black organic colorant.

According to one embodiment, the colored photosensitive resin composition may include a black organic colorant as a colorant; and optionally, may further include a black inorganic colorant and a blue colorant.

Any black inorganic colorant, any black organic colorant, and any blue colorant known in the art may be used in the present invention. For example, compounds classified as a pigment in the Color Index (published by The Society of Dyers and Colourists) and any dyes known in the art may be used.

Particular examples of the black inorganic colorant may include carbon black, titanium black, a metal oxide such as Cu—Fe—Mn-based oxide and synthetic iron black, and the like. Preferred among them is carbon black for desirable pattern properties and chemical resistance. In addition, particular examples of the black organic colorant may include aniline black, lactam black, perylene black and the like. Preferred among them is lactam black (e.g., Black 582 of BASF) for desirable optical density, permittivity, and the like. Particular examples of the blue colorant may include C.I. Pigment Blue 15:6, C.I. Pigment Blue 15:4, C.I. Pigment Blue 60, C.I. Pigment Blue 16, and the like. Preferred among them is C.I. Pigment Blue 15:6 for preventing light leakage.

The amount of the black inorganic colorant may be 0 to 20 wt %, preferably greater than 0 wt % and at most 20 wt %, and more preferably 0 to 6 wt %, based on the total solid content of the colored photosensitive resin composition (i.e., the weight excluding solvents). The amount of the black organic colorant may be 10 to 40 wt %, based on the total solid content of the colored photosensitive resin composition (i.e., the weight excluding solvents). The amount of the blue colorant may be 0 to 15 wt %, and preferably 1 to 15 wt %, based on the total solid content of the colored photosensitive resin composition (i.e., the weight excluding solvents).

The total amount of the colorant may be 10 to 60 wt %, and preferably 20 to 60 wt %, based on the total solid content of the colored photosensitive resin composition (i.e., the weight excluding solvents). Within the range, the resin composition may advantageously have a high optical density for preventing light leakage and transmittance necessary for mask alignment.

Meanwhile, a dispersing agent may be used for dispersing a colorant in the colored photosensitive resin composition of the present invention. Examples of the dispersing agent may include any known dispersing agent for a colorant. Particular examples may include a cationic surfactant, an anionic surfactant, a non-ionic surfactant, a zwitterionic surfactant, a silicon surfactant, a fluorine surfactant, and the like. Commercially available dispersing agent may include Disperbyk-182, -183, -184, -185, -2000, -2150, -2155, -2163 or -2164 from BYK Co. These compounds may be used alone or as a combination of two or more thereof. The dispersing agent may be added in advance to a colorant through surface treatment of the colorant therewith, or added together with a colorant during the preparation of a colored photosensitive resin composition.

Alternatively, the colorant may be mixed with a binder to be used for the preparation of a colored photosensitive resin composition. In this case, the binder may be the copolymer (a) described in the present invention, a known copolymer, or a mixture thereof.

Thus, the colorant used in the present invention may be added to a colored photosensitive resin composition in the form of a colored dispersion (i.e., colored mill base) obtained by mixing the colorant with a dispersing agent, a binder, a solvent, and the like.

(f) Surfactant

The colored photosensitive resin composition of the present invention may further include a surfactant to improve coatability and to prevent the generation of defects.

Although the kind of the surfactant is not particularly limited, for example, a fluorine-based surfactant or silicon-based surfactant may be used.

The commercially available silicon-based surfactant may include DC3PA, DC7PA, SH11PA, SH21PA, and SH8400 from Dowcorning Toray silicon, TSF-4440, TSF-4300, TSF-4445, TSF-4446, TSF-4460, and TSF-4452 from GE toshiba silicone, BYK 333, BYK 307, BYK 3560, BYK UV 3535, BYK 361N, BYK 354, and BYK 399 from BYK, and the like. The surfactant may be used alone or in combination of two or more thereof.

The commercially available fluorine-based surfactant may include Megaface F-470, F-471, F-475, F-482, F-489, and F-563 from DIC (Dainippon Ink Kayaku Kogyo Co.). Among them, used as the surfactant can be preferably BYK 333, and BYK 307 from BYK, and F-563 from BYK.

The amount of the surfactant may be 0.01 to 10 wt %, and preferably 0.05 to 5 wt %, based on the total solid content of the colored photosensitive resin composition (i.e., the weight excluding solvents). Within this range, the colored photosensitive resin composition may exhibit suitable coatability.

(g) Silane Coupling Agent

The colored photosensitive resin composition of the present invention may further include a silane coupling agent having a reactive substituent selected from the group consisting of carboxy, (meth)acryloyl, isocyanate, amino, mercapto, vinyl, epoxy, and a combination thereof to improve adhesiveness to a substrate, if desired.

The kind of the silane coupling agent is not specifically limited, but may preferably be selected from the group consisting of trimethoxysilyl benzoic acid, γ-methacryloxypropyltrimethoxysilane, vinyltriacetoxysilane, vinyltrimethoxysilane, γ-isocyanatopropyltriethoxysilane, γ-glycidoxypropyltrimethoxysilane, γ-glycidoxypropyltriethoxysilane, β-(3,4-epoxycyclohexyDethyltrimethoxysilane, phenylaminotrimethoxysilane, and a mixture thereof. Preferred among them is γ-isocyanatopropyltriethoxysilane having an isocyanate group (e.g., KBE-9007 from Shin-Etsu Co.) or phenylaminotrimethoxysilane, which has good chemical resistance and good adhesiveness to a substrate.

The amount of the silane coupling agent may be 0.01 to 10 wt %, and preferably 0.05 to 5 wt %, based on the total solid content of the colored photosensitive resin composition (i.e., the weight excluding solvents). Within the range, the colored photosensitive resin composition may have improved adhesiveness.

(h) Solvent

The colored photosensitive resin composition of the present invention may preferably be prepared as a liquid composition by mixing the above components with a solvent. Any solvent known in the art, which is compatible but not reactive with the components in the colored photosensitive resin composition, may be used in the preparation of the colored photosensitive resin composition.

Examples of the solvent may include glycol ethers such as ethylene glycol monoethyl ether; ethylene glycol alkyl ether acetates such as ethyl cellosolve acetate; esters such as ethyl 2-hydroxypropionate; diethylene glycols such as diethylene glycol monomethyl ether; propylene glycol alkyl ether acetates such as propylene glycol monomethyl ether acetate, and propylene glycol propyl ether acetate; and alkoxyalkyl acetates such as 3-methoxybutyl acetate. The solvent may be used alone or in combination of two or more thereof.

The amount of the solvent is not specifically limited, but may be determined so that the concentration of the solid content of the composition excluding the solvents may be 5 to 70 wt %, and preferably 10 to 55 wt %, in view of coatability and stability of a final colored photosensitive resin composition.

Further, other additives such as an antioxidant and a stabilizer may be included as long as the physical properties of a colored photosensitive resin composition are not adversely affected.

The colored photosensitive resin composition of the present invention, when formed as a cured film, may attain good height difference and may satisfy the requirements for both sensitivity and an exposure margin.

The colored photosensitive resin composition of the present invention including the above-described components may be prepared by a common method, for example, by the following method.

A colorant is mixed with a solvent in advance and dispersed therein using a bead mill until the average particle diameter of the colorant reaches a desired value. In this case, a surfactant may be used, or a portion or the whole of a copolymer may be mixed. To the dispersant thus obtained, the remainder of the copolymer and the surfactant, an epoxy resin compound or a compound derived therefrom, a polymerizable compound, and a photoinitiator are added, and an additive such as a silane coupling agent or an additional solvent, if necessary, are further mixed to a certain concentration, followed by sufficiently stirring to obtain a desired colored photosensitive resin composition.

There is also provided in the present invention a light shielding spacer formed by curing the colored photosensitive resin composition.

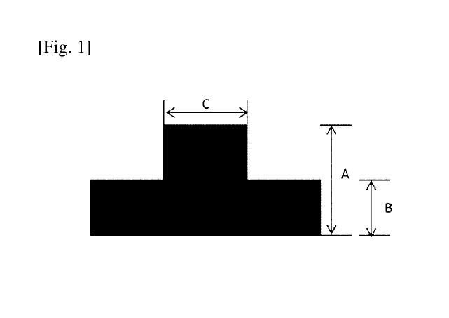

Preferably, there is provided in the present invention a black column spacer (BCS) formed using the colored photosensitive resin composition, wherein a column spacer and a black matrix are integrated into one module. An embodiment of the pattern of the black column spacer is illustrated in FIG. 1.

The column spacer, the black matrix or the black column spacer may be manufactured via a step of forming a coating, light exposing step, a developing step, and a heating step.

In the step of forming a coating, the colored photosensitive resin composition according to the present invention is coated on a pre-treated substrate by a spin coating method, a slit coating method, a roll coating method, a screen printing method, an applicator method, and the like to have a desired thickness, for example 1 to 25 μm, and then pre-cured at a temperature of 70 to 100° C. for 1 to 10 minutes and forms a coating by removing the solvent therefrom.

In order to form a pattern in the coated film, a mask having a predetermined shape is placed thereon and irradiated with an activated ray having 200 to 500 nm. In this case, in order to manufacture an integrated-type black column spacer, a mask having patterns with different transmittances may be used to accomplish a column spacer and a black matrix at the same time. As a light source used for the irradiation, a low pressure mercury lamp, a high pressure mercury lamp, an extra high pressure mercury lamp, a metal halide lamp, an argon gas laser and the like may be used; and X-ray, electronic ray and the like may also be used, if desired. The amount of light exposure may vary depending on the kind and the compositional ratio of the components of the composition and the thickness of a dried coating. When a high pressure mercury lamp is used, the amount of light exposure may be 500 mJ/cm² or less (at a wavelength of 365 nm).

After the exposing step, a developing step using an aqueous alkaline solution such as sodium carbonate, sodium hydroxide, potassium hydroxide, tetramethylammonium hydroxide, etc., as a developing solvent is performed to dissolve and remove unnecessary portions, where only an exposed portion remains to form a pattern. An image pattern obtained by the development is cooled to room temperature and post-baked in a hot air circulation-type drying furnace at a temperature of 180 to 250° C. for 10 to 60 minutes, thereby obtaining a final pattern.

The light shielding spacer thus produced may be used in the manufacture of electronic parts of an LCD, an OLED display, etc., owing to its excellent physical properties. Thus, the present invention provides an electronic part including the light shielding spacer.

The LCD, the OLED display, etc., may include any elements known to a person skilled in the art, except for the light shielding spacer according to the present invention. That is, the present invention encompasses any LCD, any OLED display, etc., in which the light shielding spacer of the present invention may be employed.

### MODE FOR THE INVENTION

Hereinafter, the present invention will be described in more detail with reference to the following examples. However, these examples are set forth to illustrate the present invention, and the scope of the present invention is not limited thereto.

### Preparation Example 1: Preparation of Copolymer

To a 500 mL, round-bottomed flask equipped with a refluxing condenser and a stirrer, 100 g of a monomer mixture having the compositional ratios described in the following Table 1, 300 g of propylene glycol monomethyl ether acetate (PGMEA) as a solvent, and 2 g of 2,2′-azobis(2,4-dimethylvaleronitrile) as a radical polymerization initiator were added, heated to 70° C. and stirred for 5 hours to obtain a copolymer solution having a solid content of 31 wt %. The copolymer thus prepared had an acid value of 100 mgKOH/g and a polystyrene-referenced weight average molecular weight (Mw) measured by gel permeation chromatography of 20,000.

TABLE 1

| | Monomers constituting copolymer (mole %) | | | | Weight |
|---|---|---|---|---|---|
| | | | 4- | | average |
| | | | hydroxybutyl | | molecular |
| | N- | | acrylate | Methacrylic | weight |
| Division | phenylmaleimide | Styrene | glycidyl ether | acid | (Mw) |
| Copolymer | 51 | 4 | 10 | 35 | 20,000 |

### Preparation Example 2: Compound Derived from Epoxy Resin Having Cardo Backbone Structure

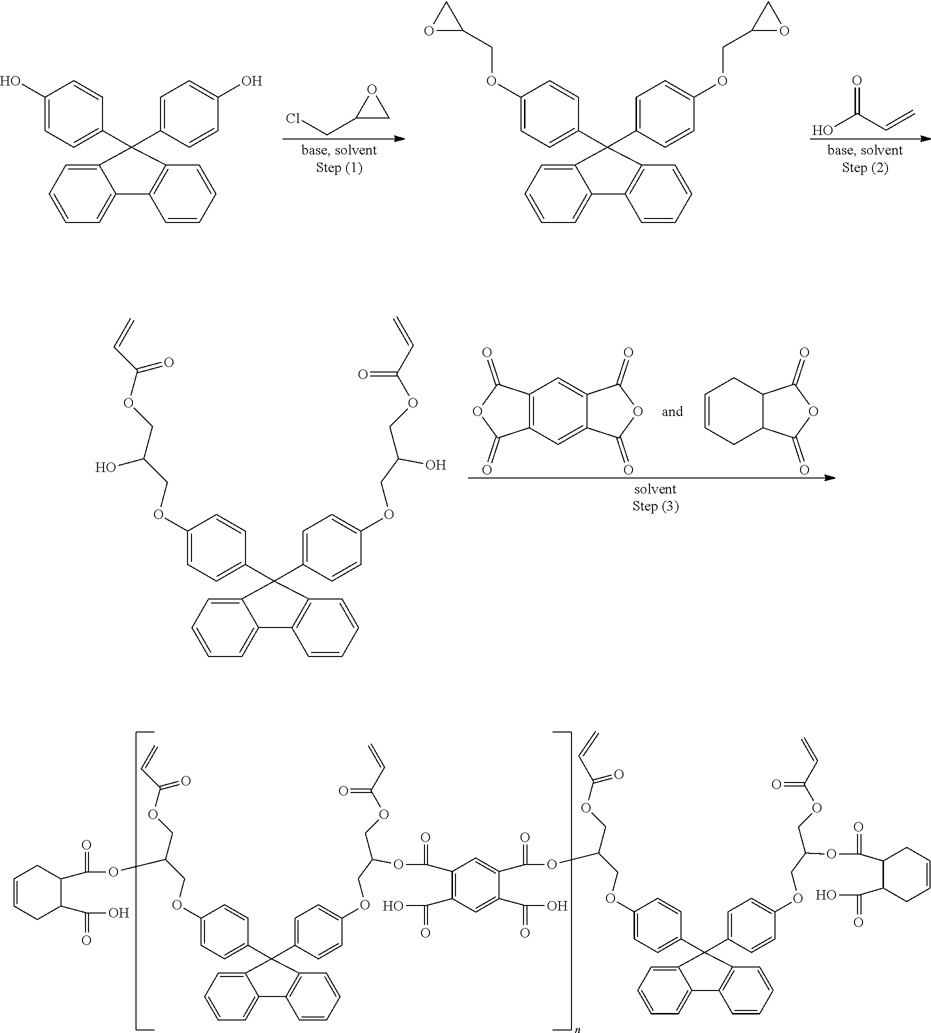

### Step (1): Preparation of 9,9-bis[4-(glycidyloxy)phenyl]fluorene

To a 3,000 mL three-neck round-bottom flask, 200 g of toluene, 125.4 g of 4,4′-(9-fluorenylidene)diphenol and 78.6 g of epichlorohydrin were added, and heated to 40° C. with stirring to obtain a solution. 0.1386 g of t-butylammonium bromide and a 50% NaOH aqueous solution (3 eq) were mixed in a vessel and the mixture was slowly added to the resulting solution with stirring.

The reaction mixture thus obtained was heated to 90° C. for 1 hour to remove 4,4′-(9-fluorenylidene)diphenol completely, which was confirmed by HPLC or TLC. The reaction mixture was cooled to 30° C., and 400 mL of dichloromethane and 300 mL of 1N HCl were added thereto with stirring. Then, the organic layer was separated, washed with 300 mL of distilled water twice or three times, dried over magnesium sulfate, and distilled under a reduced pressure to remove dichloromethane. The resultant was recrystallized using a mixture of dichloromethane and methanol to obtain the title compound, an epoxy resin compound.

### Step (2): Preparation of (((9H-fluorene-9,9-diyl)bis(4,1-phenylene))bis(oxy))bis(2-hydroxypropane-3,1-diyl) diacrylate (CAS No. 143182-97-2)

To a 1,000 mL three-neck flask, 115 g of the compound obtained in step (1), 50 mg of tetramethylammonium chloride, 50 mg of 2,6-bis(1,1-dimethylethyl)-4-methylphenol and 35 g of acrylic acid were added. The mixture was heated to 90-100° C. while blowing air at a flow rate of 25 mL/min and further heated to 120° C. to obtain a solution. The resulting solution was stirred for about 12 hours until its acid value dropped to less than 1.0 mg KOH/g and then cooled to room temperature. 300 mL of dichloromethane and 300 mL of distilled water were added to the reaction mixture with stirring. Then, the organic layer was separated, washed with 300 mL of distilled water twice or three times, dried over magnesium sulfate, and distilled under a reduced pressure to remove dichloromethane, thereby providing the title compound.

Step (3): Preparation of a Compound Derived from an Epoxy Resin Compound Having a Cardo Backbone Structure

The compound obtained in step (2) in PGMEA was placed into a 1,000 mL three-neck flask, and 1,2,4,5-benzenetetracarboxylic acid dianhydride (0.75 eq), 1,2,3,6-tetrahydrophthalic acid anhydride (0.5 eq) and triphenylphosphine (0.01 eq) were further added thereto. The reaction mixture was heated to 120-130° C. for 2 hours with stirring and then cooled to 80-90° C., followed by stirring for 6 hours. After cooling to room temperature, a solution (solid content of 49 wt %) of polymer having a weight average molecular weight (Mw) of 6,000 and an acid value of 107 mg KOH/g (based on the solid content) was obtained.

### Preparation Example 3: Preparation of Colored Dispersion

8 g of the copolymer solution obtained in Preparation Example 1 above, 8 g of a polymer dispersing agent (DISPERBYK-2000, BYK), 12 g of carbon black, 53 g of lactam black (Black 582, BASF) as an organic black, 16 g of C.I. Pigment Blue 15:6, and 384 g of PGMEA as a solvent were placed in a painter shaker, and the mixture was dispersed at 25 to 60° C. for 6 hours. This dispersing step was performed with 0.3 mm zirconia beads. Upon completion of the dispersion, the beads were separated from the dispersion using a filter, thereby obtaining a colored dispersion having a solid content of 23 wt %.

### Example 1: Preparation of Colored Photosensitive Resin Composition

(a) 7.7 g of the copolymer solution obtained in Preparation Example 1, (b) 7.5 g of the polymer solution obtained in Preparation Example 2, (c) 4.3 g of dipentaerythritol hexaacrylate (DPHA, Nippon Kayaku) as a polymerizable compound, (d-1) 0.205 g of an oxime photoinitiator represented by Formula 1b (N-1919, ADEKA), (d-2) 0.205 g of a triazine initiator represented by Formula 2a (T-Y, PHARMASYNTEHSE), (e) 36.0 g of the colored dispersion prepared in Preparation Example 3 as a colorant, and (f) 0.009 g of a surfactant (BYK-307, BYK) were added to 44.0 g of a PGMEA solvent, followed by mixing and stirring for 5 hours according to a conventional method to obtain a colored photosensitive resin composition.

### Examples 2 to 5 and Comparative Examples 1 to 6: Preparation of Colored Photosensitive Resin Compositions

Colored photosensitive resin compositions were prepared by the same procedure described in Example 1 except for changing the amount of the photoinitiators as illustrated in the following table 2.

TABLE 2

| | Oxime | Triazine |
|---|---|---|
| | photoinitiator (d-1) | photoinitiator (d-2) |
| Example 1 | 0.205 g | 0.205 g |
| Example 2 | 0.241 g | 0.241 g |
| Example 3 | 0.277 g | 0.277 g |
| Example 4 | 0.272 g | 0.205 g |
| Example 5 | 0.205 g | 0.272 g |
| Comparative Example 1 | 0.000 g | 0.401 g |
| Comparative Example 2 | 0.000 g | 0.471 g |
| Comparative Example 3 | 0.000 g | 0.532 g |
| Comparative Example 4 | 0.482 g | 0.000 g |
| Comparative Example 5 | 0.410 g | 0.000 g |
| Comparative Example 6 | 0.224 g | 0.000 g |

### Experimental Example 1: Manufacture of Cured Film from Colored Photosensitive Resin Composition

The colored photosensitive resin compositions obtained in the examples and comparative examples were coated on glass substrates using a spin coater and pre-baked at 80° C. for 150 seconds to form coated films. On the coated film thus formed, a pattern mask composed of a 100% full-tone column spacer (CS) pattern and a 20% half-tone black matrix pattern was applied, and irradiated with light having a wavelength of 365 nm in the amount of light exposure of 40 mJ/cm². After checking the break point (BP) time at 23° C., development was performed using a 0.04 wt % aqueous solution of potassium hydroxide for additional 15 seconds, followed by washing with pure water for 1 minute. The pattern thus formed was post-baked in an oven at 230° C. for 30 minutes to obtain each cured film (light shielding spacer).

### Experimental Example 2: Evaluation of Sensitivity (Measurement of Central Exposure Energy)

During manufacturing cured films using the compositions of the examples and comparative examples according to the procedure described in Experimental Example 1, light exposure energy (mJ/cm²) for obtaining a film thickness (i.e., a thickness corresponding to B in FIG. 1) of 2.0 μm by applying a 20% half-tone mask was measured. The light exposure energy thus measured of 65 mJ/cm² or less was preferable in consideration of sensitivity.

### Experimental Example 3: Measurement of Exposure Margin

During manufacturing cured films using the compositions of the examples and comparative examples according to the same procedure described in Experimental Example 1, the film thickness obtained by applying a 20% half-tone mask was measured. Specifically, each composition was exposed to a greater amount of energy by 3.5 mJ than its central exposure energy, and the film thickness (μm) thus obtained was measured (“T_{E+3.5}”). Separately, each composition was exposed to a smaller amount of energy by 3.5 mJ than the central exposure energy, and the film thickness (μm) thus obtained was measured (“T_{E-3.5}”). The film thickness was measured using a non-contact type optical device (SNU precision). The exposure margin was computed using the measured film thicknesses based on the following formula.

Exposure margin (μm/mJ)=[T_{E+3.5} (μm)−T_{E-3.5} (μm)]/7.0 mJ

The exposure margin thus measured of 0.10 μm/mJ or less was preferable.

The results of Experimental Examples 2 and 3 are summarized in the following Table 3.

TABLE 3

| | Central exposure energy | Exposure margin |
|---|---|---|
| Division | (mJ/cm²) | (μm/mJ) |
| Example 1 | 56 | 0.08 |
| Example 2 | 52.5 | 0.09 |
| Example 3 | 45.5 | 0.09 |
| Example 4 | 45.5 | 0.10 |
| Example 5 | 59.5 | 0.09 |
| Comparative Example 1 | 157.5 | 0.06 |
| Comparative Example 2 | 143.5 | 0.06 |
| Comparative Example 3 | 133.5 | 0.06 |
| Comparative Example 4 | 23.5 | 0.225 |
| Comparative Example 5 | 28.4 | 0.195 |
| Comparative Example 6 | 54.5 | 0.145 |

As shown in Table 3, the cured films manufactured using the colored photosensitive resin compositions of Examples 1 to 5 had the central exposure energy of 65 mJ/cm² or less and the exposure margin of 0.10 μm/mJ or less, which indicated good sensitivity and exposure margin. The compositions according to the examples exhibited reliability in manufacturing a black column spacer having a height difference.

On the contrary, the cured films manufactured using the colored photosensitive resin compositions of Comparative Examples 1 to 3 had the central exposure energy exceeding 65 mJ/cm² and the cured films manufactured using the colored photosensitive resin compositions of Comparative Examples 4 to 6 had the exposure margins exceeding 0.10 μm/mJ. Thus it was found that the cured films manufactured in the comparative examples were deteriorated when compared to those of the examples in terms of the sensitivity and the exposure margin.

1. A colored photosensitive resin composition, which comprises:
(a) a copolymer;
(b) an epoxy resin compound or a compound derived therefrom;
(c) a polymerizable compound;
(d) a photoinitiator; and
(e) a colorant,
wherein the photoinitiator comprises a compound of the following Formula 1 and a compound of the following Formula 2: 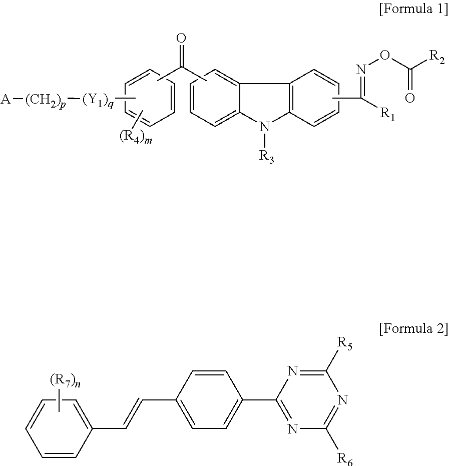
wherein, in Formula 1, R₁ to R₄ are each independently hydrogen, deuterium, halogen, substituted or unsubstituted C₁₋₁₂ alkyl, substituted or unsubstituted C₂₋₁₂ alkenyl, substituted or unsubstituted halo-C₁₋₁₂ alkyl, substituted or unsubstituted C₆₋₁₂ aryl, substituted or unsubstituted C₃₋₁₂ cycloalkyl, substituted or unsubstituted C₁₋₁₂ alkoxy, or C₁₋₁₂ ester; A is substituted or unsubstituted 5- to 12-membered heteroaryl, or substituted or unsubstituted 5- to 7-membered heterocycloalkyl; R₁ to R₄ and substituents of A are each independently at least one selected from the group consisting of halogen, halo-C₁₋₁₂ alkyl, C₁₋₁₂ alkyl, C₂₋₁₂ alkenyl, C₆₋₁₂ aryl, C₃₋₁₂ cycloalkyl, C₁₋₁₂ alkoxy, carboxy, nitro, and hydroxy; Y₁ is —O—, —S—, or —Se—; m is an integer of 0 to 4; in the case where m is an integer of 2 or more, R₄s are the same or different from each other; p is an integer of 0 to 5; and q is 0 or 1, and
in Formula 2, R₅ and R₆ are halomethyl; R₇ is each independently C₁₋₄ alkyl or C₁₋₄ alkoxy; and n is an integer of 0 to 3.

2. The colored photosensitive resin composition of claim 1, wherein the compound of Formula 1 is represented by the following Formula 1a: 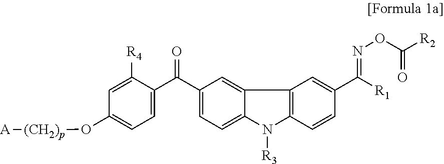
wherein, in Formula 1a, R₁ to R₄, p and A are the same as defined in claim 1.

3. The colored photosensitive resin composition of claim 2, wherein the compound of Formula 1 is represented by the following Formula 1b:

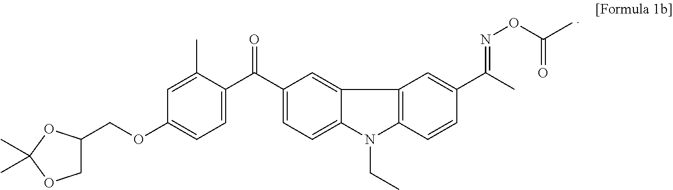 [SMILES: CCn1c2ccc(C(=O)c3ccc(OCC4COC(C)(C)O4)cc3C)cc2c2cc(/C(C)=N/OC(C)=O)ccc21 ; "."]

4. The colored photosensitive resin composition of claim 1, wherein the compound of Formula 2 is represented by the following Formula 2a:

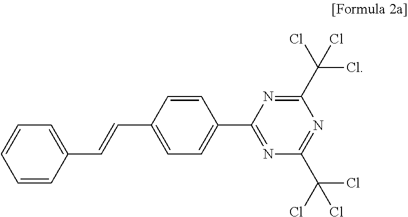 [SMILES: ClC(Cl)(Cl)c1nc(-c2ccc(/C=C/c3ccccc3)cc2)nc(C(Cl)(Cl)Cl)n1]

5. The colored photosensitive resin composition of claim 1, wherein the photoinitiator comprises the compound of Formula 1 and the compound of Formula 2 in a weight ratio of 8:2 to 2:8.

6. The colored photosensitive resin composition of claim 1, wherein the epoxy resin compound or the compound derived therefrom has a cardo backbone structure.

7. The colored photosensitive resin composition of claim 1, wherein the colorant comprises:
0 to 20 wt % of a black inorganic colorant;
10 to 40 wt % of a black organic colorant; and
0 to 15 wt % of a blue colorant,
based on the total solid content of the colored photosensitive resin composition.

8. A light shielding spacer formed by curing the colored photosensitive resin composition described in any one of claims 1 to 7.
